(19) United States
(12) Patent Application Publication
(10) Pub. No.: US 2023/0404480 A1
(43) Pub. Date: Dec. 21, 2023

(54) EAR TIP, EAR TIP COMPONENT, AND EARPHONE

(71) Applicant: VIE STYLE, INC., Kanagawa (JP)

(72) Inventor: Yasuhiko IMAMURA, Kanagawa (JP)

(21) Appl. No.: 18/035,499

(22) PCT Filed: Nov. 5, 2021
(86) PCT No.: PCT/JP2021/040807

§ 371 Date: May 4, 2023

(30) Foreign Application Priority Data
Nov. 6, 2020 (JP) 2020-185873

(51) Int. Cl. A61B 5/00; H04R 1/10

(52) U.S. Cl. CPC A61B 5/6803; H04R 1/1016; H04R 2420/07

(57) ABSTRACT

This ear tip includes: a conductive first member (10) having a cylindrical portion (11) having a first end and a second end, and a contact portion extending from the first end to the second end side; and an elastic second member (20) covering the outside of the cylindrical portion, wherein the contact portion is located on the outside of the second member.

### TECHNICAL FIELD

The present invention relates to an ear tip, an ear tip component, and an earphone.

### BACKGROUND ART

There has conventionally been known a technique that uses silver as a conductive agent in the ear tips of earphones that acquire biological signals (see, for example, Patent Documents 1 and 2).

### CITATION LIST

### Patent Document

Patent Document 1: Patent Publication JP-A-2014-215963
Patent Document 2: Patent Publication JP-A-2019-24758

### SUMMARY

### Technical Problem

In the ear tips of the prior art, silver is sometimes used as a conductive filler mixed with rubber, to increase conductivity. However, since silver is expensive, mixing a silver filler in an entire ear tip increases the cost of the ear tip. Furthermore, a plurality of sizes of ear tips may be prepared for each user with various ear shapes and ear canals, and preparing a plurality of ear tips with silver filler for one user further increases the cost.

Therefore, one aspect of the present invention is to provide an ear tip capable of properly acquire biological signals while reducing costs.

### Solution to Problem

An ear tip according to one aspect of the present invention includes: a conductive first member having a cylindrical portion having a first end and a second end, and a contact portion extending from the first end to the second end side; and an elastic second member covering the outside of the cylindrical portion, wherein the contact portion is located on the outside of the second member.

### Advantageous Effects of Invention

According to one aspect of the present invention, an ear tip capable of properly acquiring biological signals while reducing costs can be provided.

### BRIEF DESCRIPTION OF DRAWINGS

FIG. 1 is a diagram showing an example of the entire earphone according to a first embodiment.

FIG. 2 is a diagram showing an example of the appearance of an ear tip in a YZ plane viewed from an X direction according to the first embodiment.

FIG. 3 is a diagram showing an example of a cross section passing through a central axis R in a Z direction of a first member according to the first embodiment.

FIG. 4 is a diagram showing an example of the appearance of the first member in an XZ plane viewed from a Y direction according to the first embodiment.

FIG. 5 is a diagram showing an example of the ear tip where another second member is used according to the first embodiment.

FIG. 6A is a diagram showing an example of a first member according to a modification.

FIG. 6B is a diagram showing an example of an ear tip according to the modification.

FIG. 7A is a diagram showing an example of the appearance of an ear tip 1D in the YZ plane according to the modification.

FIG. 7B is a perspective view of the ear tip 1D according to the modification.

FIG. 8 is a diagram showing an example of an earphone according to a second embodiment.

FIG. 9 is a diagram showing an example of the earphone according to the second embodiment.

FIG. 10 is a diagram showing an example of a ground sensor according to the second embodiment.

FIG. 11 is a diagram showing an example of a reference sensor according to the second embodiment.

FIG. 12 is a diagram for explaining how three sensors of the earphone according to the second embodiment come into contact with a wearer.

FIG. 13 is a diagram for explaining an overview of the earphone according to the second embodiment.

### DESCRIPTION OF EMBODIMENTS

Embodiments of the present invention are now described hereinafter with reference to the drawings. However, the embodiments described below are merely exemplary and are not intended to exclude the application of various modifications and techniques not explicitly described below. That is, the present invention can be implemented with various modifications to the extent not departing from the gist of the present invention. In the following drawings, identical or similar parts are indicated with identical or similar symbols. The drawings are schematic and do not necessarily correspond to actual dimensions and proportions. The drawings may include portions where the relationship of dimensions and proportions differ from each other even among the drawings.

### First Embodiment

The following describes an overview of an earphone according to a first embodiment, followed by an example of an ear tip of the first embodiment, each illustrated with drawings.

<Overview of Earphone>

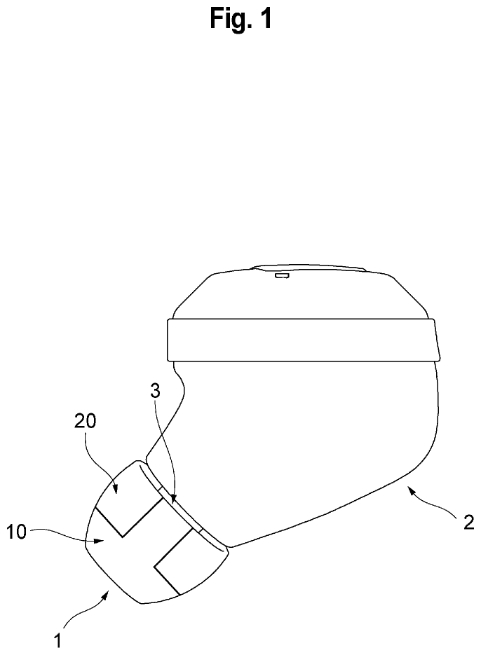

First the overview of the earphone according to the first embodiment is described. FIG. 1 is a diagram showing an example of the entire earphone according to the first embodiment.

The earphone shown in FIG. 1 is one of a pair of earphones. The earphone includes an ear tip 1 and an earphone body portion 2. The earphone body portion 2 includes a nozzle (connection portion) 3 that removably attaches the ear tip 1 and the earphone body portion 2. The nozzle 3 is, for example, a portion that constitutes a sound conductor, has electrodes, and has a contact point that electrically connects to a conductive portion of the ear tip 1, which will be described later. The nozzle 3 also has a mounting structure detachable with respect to the ear tip 1.

The earphone body portion 2 can include, for example, a communication circuit (communication interface) that communicates sound signals to other devices, an operation unit functioning to operate the earphone, a power source (battery), a microphone, and the like. In the example shown in FIG. 1, the earphone is shown as a wireless type, but the earphone may have a cable that includes a plurality of signal lines connecting each circuit and the like in the earphone. The earphone body portion 2 also has a biosensor that acquires biological signals detected from the ear tip 1. The biological signals are, for example, brain wave signals, eye potential signals, and the like, and in the following, EEG signals are used as an example.

In the example of the ear tip 1 shown in FIG. 1, the ear tip 1 is formed by a first member (component) 10 and a second member 20 that have conductivity. For example, the first member 10 and the second member 20 are formed from different materials and are each detachable. The shape of the first member 10 is not limited to the example shown in FIG. 1, as long as it has a portion that comes into contact with an inner wall of the ear canal of the wearer and this contact portion is in proper contact with the ear canal. The larger the surface area of this contact portion, the better. It is preferred that the second member 20 be formed of an inexpensive non-conductive elastic material (e.g., silicone rubber).

<Overview of Ear Tip>

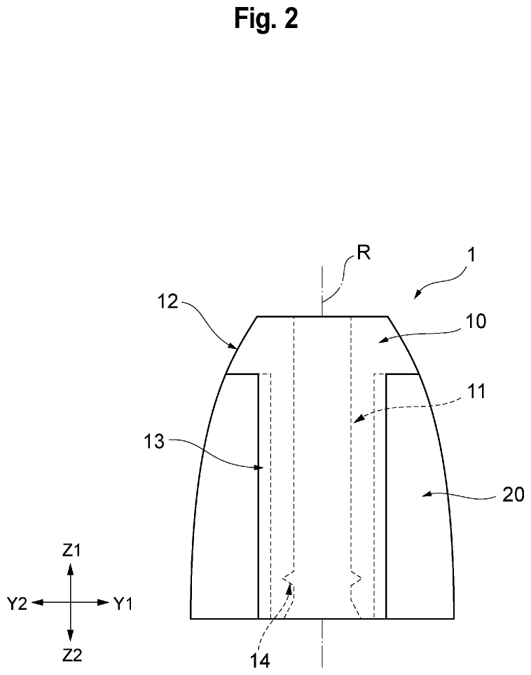

FIG. 2 is a diagram showing an example of the appearance of the ear tip 1 in a YZ plane viewed from an X direction according to the first embodiment. The ear tip 1 shown in FIG. 2 includes the first member 10 located on the eardrum side (Z1 direction) and the second member 20 located on the earphone body portion 2 side (Z2 direction). The first member 10 is made of, for example, conductive rubber, which contains silver or silver chloride. Preferably, in order to ensure adequate conductivity, the silver or silver chloride contains 10% by mass or more of the conductive material contained in the conductive rubber.

The first member 10 may also be formed of a silicon material containing a metallic filler. For example, the first member 10 can be made highly conductive by appropriately blending silver, copper, gold, aluminum, zinc, nickel, and the like into the silicon material as metallic fillers. It is not necessary that all of the filler contained be silver or silver chloride, but only that a part of the filler be silver or silver chloride. This reduces the silver or silver chloride content, thus reducing the hardness of the rubber and creating conductive rubber having moderate hardness.

The first member 10 shown in FIG. 2 includes a cylindrical portion 11 having a second end in the Z2 direction and a first end in the Z1 direction (dotted portion shown in FIG. 2), and a contact portion (including a first portion 12 and a second portion 13) extending from the first end of the cylindrical portion 11 toward the second end. The contact portion includes the first portion (tip) 12 that is formed into a dome (or a bowl) shape from the first end and the second portion (extended portion) 13 that extends from a predetermined position of this tip 12 to the second end side.

The cylindrical portion 11 is hollow inside, and this cavity serves as a sound conductor for the sound to be output from the earphone body portion 2. The sound to be output by the earphone body portion 2 and through the nozzle passes from the second end of the cylindrical portion 11 through the cavity at the first end and reaches the eardrum of the wearer. The cylindrical portion 11 does not necessarily have to be shaped into a hollow cylinder, as long as it is configured to form a sound conductor.

The first portion (tip) 12 of the contact portion has, for example, a dome or bowl shape with a concave portion, and an opening at the first end of the cylindrical portion 11 is formed at the center portion of the bowl-shaped bottom. A convex direction of the tip 12 is located on the first end side. The tip 12 is formed so that the radius from the central axis R becomes shorter as the tip 12 extends in the Z1 direction (toward the first end side) in order to facilitate the entry of the tip 12 into the ear canal when the ear tip 1 is placed in the ear canal. The tip 12 does not necessarily have to be dome-shaped or bowl-shaped, but can be shaped to facilitate the insertion thereof into the ear along the ear canal (i.e., the diameter decreases toward the tip).

The second portion of the contact portion (extended portion) 13 is a portion extending from the first end side of the cylindrical portion 11 to the second end side, and is formed on the outside of the cylindrical portion 11. For example, the extended portion 13 has a planar shape extending from the first end side to the second end side of the cylindrical portion and at least partially contacts the inner wall of the wearer's ear canal. The extended portion 13 may be formed, for example, by at least a part of the tip 12 extending toward the second end portion, the tip 12 being located at the first end of the cylindrical portion 11.

The extended portion 13 is not limited to the example shown in FIG. 2; at least one or more of the extended portions 13 may be provided in the first member 10. In the example shown in FIG. 2, the extended portion 13 is also provided on the back side, and two of the extended portions 13 are provided on the first member 10. In the example shown in FIG. 2, the extended portions 13 have a plate-like shape, but the shape is not limited to this plate-like shape and may include a flat surface that appropriately comes into contact with the inner wall of the ear canal of the wearer. It is preferred that the extended portions 13 have as large a surface area as possible. The extended portions 13 need not be straight in the Z direction or Y direction but may have a rounded curved surface to fit along a curve of an outer surface of the second member 20, which will be described later.

A mounting structure 14 (indicated by dotted lines) has a structure or mechanism that can be attached to and detachable from the earphone body portion 2. The mounting structure 14 has, for example, a concave portion in a circumferential direction on the second end side of the cylindrical portion 11. The mounting structure 14 including this concave portion is removably fitted into a convex portion formed on the nozzle 3 of the earphone body portion 2. The convex portion may be provided on the cylindrical portion 11, and the concave portion may be provided on the nozzle 3. In addition to using the concavity and convexity, any mechanism or structure may be used for the mounting structure 14, as long as it is a mechanism or structure that can be attached and detached. The mounting structure 14 may employ an ear tip detachable structure or mechanism that is employed in known earphones and the like.

According to the first embodiment described above, by mixing the metallic filler in the first member 10 constituting a part of the ear tip 1, the price at the time of sale can be reduced compared to mixing the metallic filler in the entire ear tip 1.

As in the prior art, if a silicon material mixed with metallic fillers (silver, copper, gold, aluminum, zinc, nickel, etc.) is employed for the entire ear tip, increasing the amount of carbon-based particles in order to lower the impedance is considered. However, if the amount of carbon-based particles is increased too much, the resilience will be weakened, the ear tip surface will be easily damaged by tearing or the like, and the pressure on the skin will be low. As a result, it becomes difficult to acquire good quality biological signals.

In addition, if more carbon-based particles are added to the entire ear tip, which is made of silicon material mixed with a metallic filler (silver, copper, gold, aluminum, zinc, nickel, etc.), and the hardness of the silicon is increased to prevent breakage, the ear tip will be uncomfortable to wear and will not fit the shape of the ear.

Therefore, in the first embodiment, the following configuration is adopted for the first member 10 as an example in order to make the material have moderate flexibility while lowering the impedance.
For the silicon material mixed with AG (silver) filler, the carbon content is 10% or more.
The impedance of the silicon material mixed with AG filler is, for example, 1×105 Ω·cm or less in volume specific low efficiency.
The hardness of the silicon material mixed with AG filler is 30 to 50° (degrees) or less.

By using a silicon material mixed with AG filler as described above, the material can be made moderately flexible and easily fits into the ear canal when worn. Also, by moderately mixing the AG filler, the impedance can be lowered and appropriate biological signals, such as EEG signals, can be acquired.

The first member 10 may be created by integrally molding the cylindrical portion 11 and the contact portion including the tip 12 and the extended portions 13 using a mold or the like, or may be made of the same conductive silicone material described above. Even if the cylindrical portion 11 and the contact portion are separate members, the cylindrical portion 11 and the contact portion may be mutually conductive and connected. Since the cylindrical portion 11 is the sound conductor, it is preferred that the shape thereof not be deformed so as not to block the cavity. Therefore, while the cylindrical portion 11 is conductive, the hardness thereof may be greater (harder) than the hardness of the extended portion 13. Since the tip 12 is also a part that comes into contact with the ear canal, the hardness of the cylindrical portion 11 may be greater than the hardness of the tip 12.

The second member 20 is an elastic member that approximately determines the overall size of the ear tip 1. The second member 20 is, for example, rubber, and is formed of a material employed for normal ear tips. The second member 20 has a shape that covers the cylindrical portion 11 of the first member 10, which is, for example, a hollow donut shape or a hollow cylinder.

Since the second member 20 is hollow, the second member 20 is attached to the first member 10 by sliding and inserting the cylindrical portion 11 into the cavity of the second member 20, from the second end of Z2 toward the first end of Z1. As a result, the second member 20 is easily attached to and detached from the first member 10. In so doing, the tip portion of the second member 20 in the Z1 direction may be stored in the space of the tip 12 of the first member 10. In this case, the tip portion of the second member is housed in the space of the tip 12 of the first member 10, so that the cylindrical portion 11 cannot slide farther and is less likely to shift in the horizontal direction (X direction or Y direction). The method of attaching and detaching the first member 10 and the second member 20 is not limited to the example described above.

The size of the second member 20 is determined by the length of the radius from the central axis to the outside in the Y direction. For example, in the Z direction, three average radius lengths in the Y direction may be provided, and the sizes thereof may be L, M, and S, starting from the largest. Even with the same size of the second member 20, the radius in the Y direction may be shortened from the Z2 direction toward the Z1 direction (the tip of the ear tip 1).

The extended portion 13 of the first member 10 is elastic and can be elastically deformed outwardly by the second member 20. For example, when this ear tip 1 is inserted into an ear canal slightly narrower than the diameter around the central axis R of the ear tip 1, the elastic second member 20 is inserted with pressure in the outward direction. This allows the extended portion 13, which is located between the second member 20 and the ear canal, to properly come into contact with the inner wall of the ear canal as the second member 20 exerts pressure against the ear canal.

In addition, for the extended portion 13, a distance L1 from the central axis R gradually increases, from the first end side toward the second end side. Also, for the second member 20, a distance L2 from the central axis R to an outer edge gradually increases, from the first end side to the second end side. In this case, at least a part of the extended portion 13 may be L1<L2. As a result, when the second member 20 is attached to the first member 10, the extended portion 13 is pressured outwardly in the Y direction by the second member 20, and the extended portion 13 is pushed outwardly in the Y direction. As a result, as described above, when the ear tip 1 is inserted into the ear canal, the extended portion 13 can come into contact with the inner wall of the ear canal more appropriately by the pushing force from the second member 20 toward the ear canal and the reaction force from the ear canal pushing back.

With at least the extended portion 13 properly coming into contact with the ear canal, the contact portion detects a biological signal (e.g., an EEG signal), and this biological signal is conducted through the cylindrical portion 11. The biological signal is then conducted from the contact point of the earphone body portion 2 to the biosensor of the earphone body portion 2. This allows the biosensor of the earphone body portion 2 to properly acquire biological signals to be conducted through the first member 10.

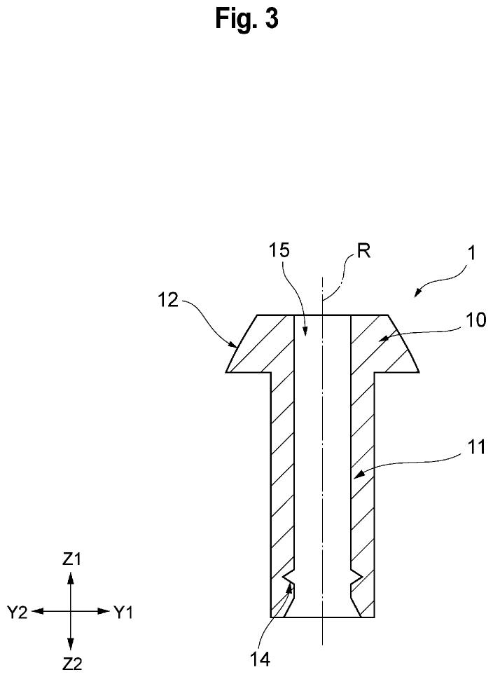

FIG. 3 is a diagram showing an example of a cross section passing through the central axis R in the Z direction of the first member 10 according to the first embodiment. As shown in FIG. 3, the cylindrical portion 11 has a hollow portion 15 passing through the central axis R, and this hollow portion 15 serves as a sound path for conducting sound. The cylindrical portion 11 includes the mounting structure 14 in the vicinity of the second end in the Z2 direction, and the first member 10 and the second member 20 are attached by fitting the convex portion provided on the nozzle 3 of the earphone body portion 2 into the concave portion of the mounting structure 14.

For example, an electrode is provided on the nozzle 3 of the earphone body portion 2, and a contact point of this electrode make contact near the second end of the cylindrical portion 11. As an example, the convex portion on the nozzle 3 of the earphone body portion 2 is fitted into the concave portion of the mounting structure 14, so that the cylindrical portion 11 and the contact point of the nozzle 3 are in proper contact.

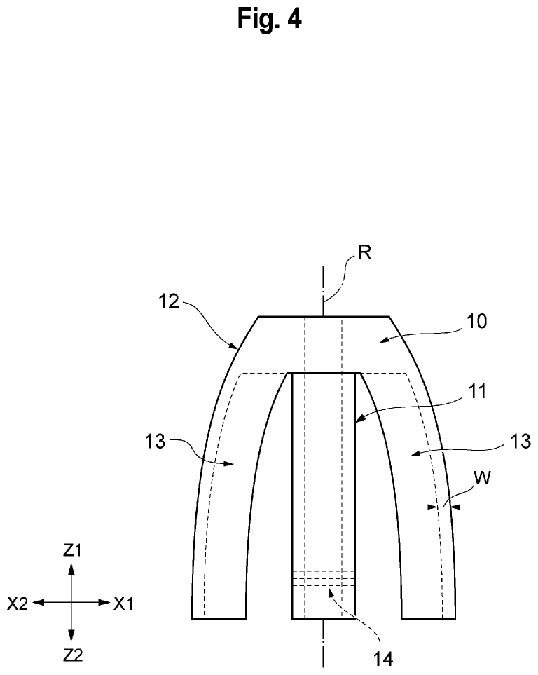

FIG. 4 is a diagram showing an example of the appearance of the first member in an XZ plane viewed from the Y direction according to the first embodiment. In the example shown in FIG. 4, the contact portions (the tip 12 and the extended portion 13) are provided on the outside of the first member 10 in the X1 direction and the X2 direction. The cylindrical portion 11 with a cavity containing the central axis R in the Z direction is provided at the center of the first member 10. The thickness of the extended portion 13 is W. The extended portion 13 has a rounded curved shape in the XZ plane and also in an XY plane. For example, the extended portion 13 may be formed by being folded back and stretched from the first end of the cylindrical portion 11 including an opening in the Z1 direction.

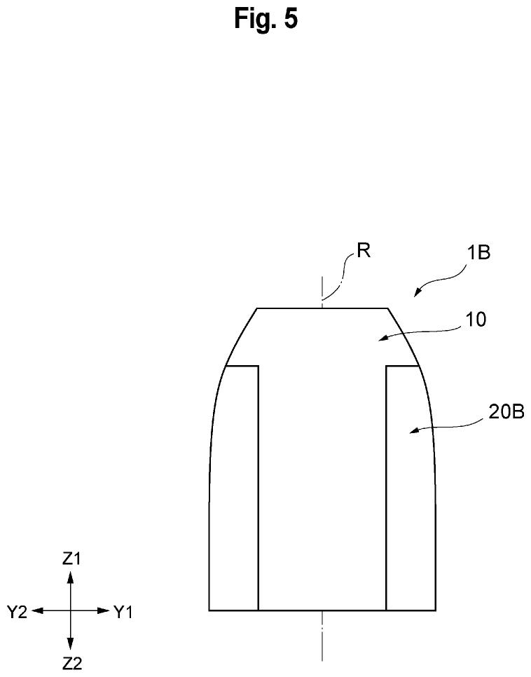

FIG. 5 is a diagram showing an example of an ear tip 1B where another second member 20B is used according to the first embodiment. The second member 20B in the example shown in FIG. 5 is smaller in diameter and size than the second member 20 shown in FIG. 2. That is, the surface volume of the second member 20B is smaller than the surface volume of the second member 20 because the average diameter of the second member 20B in the XY plane is shorter than the average diameter of the second member 20 in the same plane. As shown in FIG. 5, it is possible to removably mount second members of different sizes to the common first member 10.

This allows each user to change the size of the ear tip itself by selecting one second member from a plurality of second members and combining said second member with the common first member, according to the size and shape of his/her ear canal.

As described above, the ear tip 1 according to the first embodiment has a two-stage structure with the first member 10 and the second member 20 in order to ensure sufficient conductivity and at the same time to have a structure with appropriate hardness that changes appropriately in the ear. In order to detect a biological signal with the first member 10, the first member 10 may be made of a conductive elastic electrode containing a metallic (e.g., AG) filler, and the second member 20 may be made of inexpensive elastic rubber in order to reduce the price of the ear tip.

The second member 20 may be less hard (more flexible) than the first member 10. This makes it easier to insert the ear tip into the ear canal by using the flexibility of the second member 20, which forms part of the ear tip, to conform to the shape of the ear canal when the ear tip is inserted into the ear canal.

The first member 10 and the second member 20 may be removably mounted. This makes it possible to use the expensive first member 10 as a common component and change the inexpensive second member 20 accordingly, thereby making the size of the ear tip itself variable and also reducing the selling cost.

By providing the first member 10 with the cylindrical portion 11, the tip 12, and the extended portion 13, the extended portion 13 and the tip 12 are pressed (crimped) against the inner wall of the ear canal and can detect a biological signal with high accuracy. In addition, the cylindrical portion 11, the tip 12, and the extended portion 13 can be integrally molded, and if they are integrally molded, manufacturing costs can be reduced.

[Modifications]

The first embodiment of the technique disclosed in the present application has been described above, but the technique disclosed in the present application is not limited to the above.

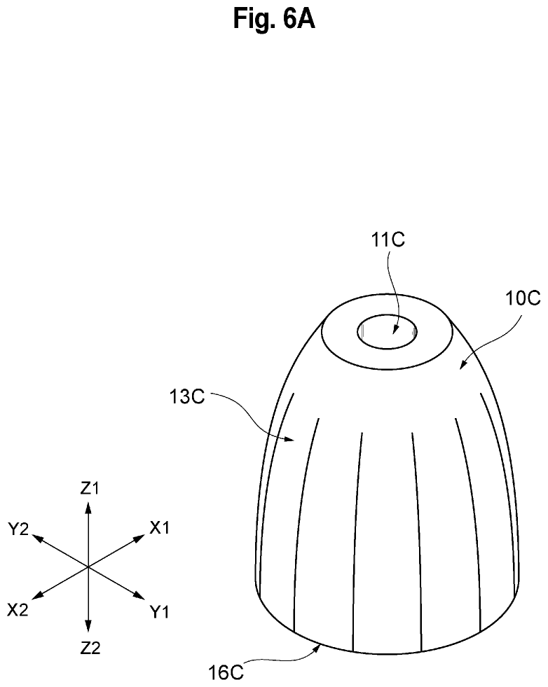

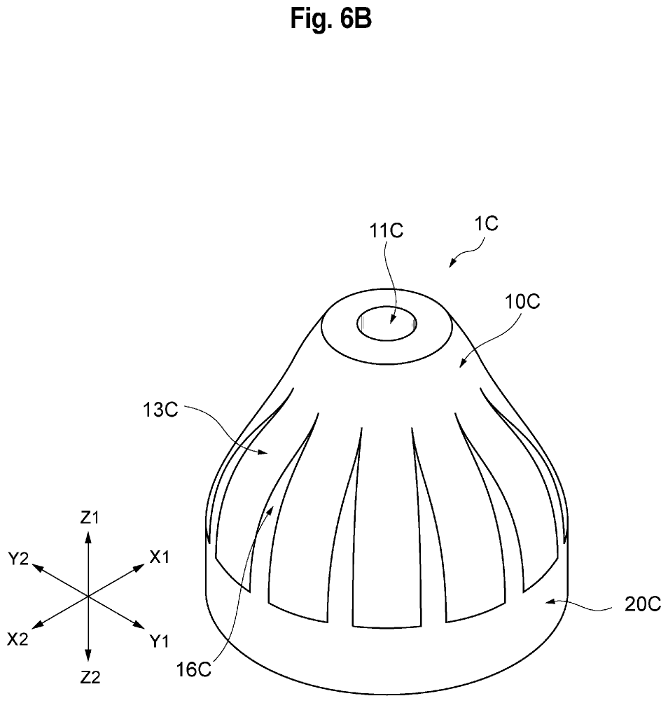

FIG. 6A is a diagram showing an example of a first member 10C according to a modification. In the example shown in FIG. 6A, a plurality of extended portions 13C of the first member 10C are coupled to a first end via a dome-shaped tip and are formed radially from a predetermined position of this tip. As a result, the plurality of extended portions 13C are radially formed so as not to overlap each other, and the surface area in contact with the inner wall to the ear canal can be increased, enabling the acquisition of biological signals with high accuracy.

On the second end side of the plurality of extended portions 13C, a slit 16C may be provided between two extended portions 13C. For example, the slit 16C is provided along the Z-direction (central axis direction). This slit allows the extended portions 13C to open and close radially as needed, in accordance with the size of the second member 20.

FIG. 6B is a diagram showing an example of an ear tip 1C according to the modification. In the example shown in FIG. 6B, a second member 20C is inserted into the cylindrical portion of the first member 10C. As shown in FIG. 6B, a slit 16C is radially spread out so that contact portions of the first member 10C (e.g., the extended portions 13C) can properly come into contact with an outer peripheral surface of the second member 20C, in accordance with the size of the second member 20C.

The radial shape of the first member 10C shown in FIG. 6 is an example and is not limited to this shape. In order to increase the area in contact with the inner wall of the ear canal as much as possible, the number of slits 16C is preferably as few as possible.

The first member 10 may also have at least two extended portions 13 and insulate the respective conduction paths to the corresponding contact points of the nozzle, so that one of them is obtained as a reference signal. In this case, the cylindrical portion 11 of the first member 10 may be divided into two regions by dividing it in a plane through the central axis, and each region may be insulated.

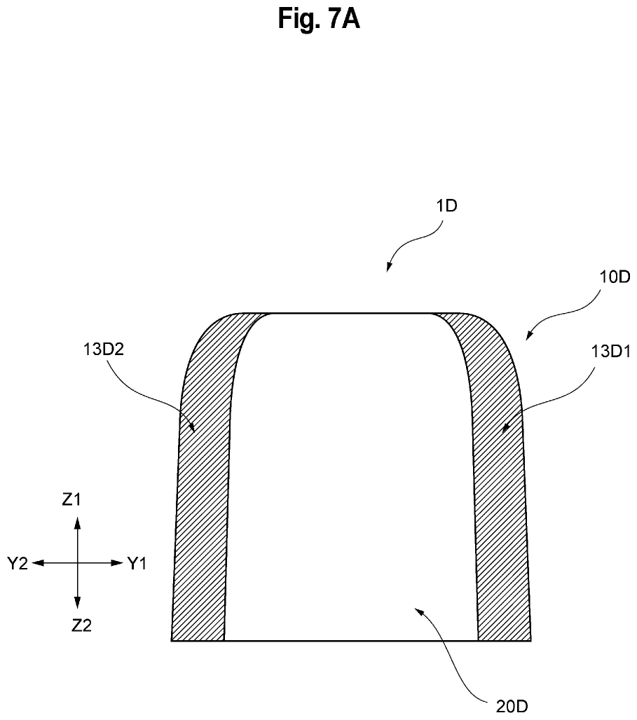

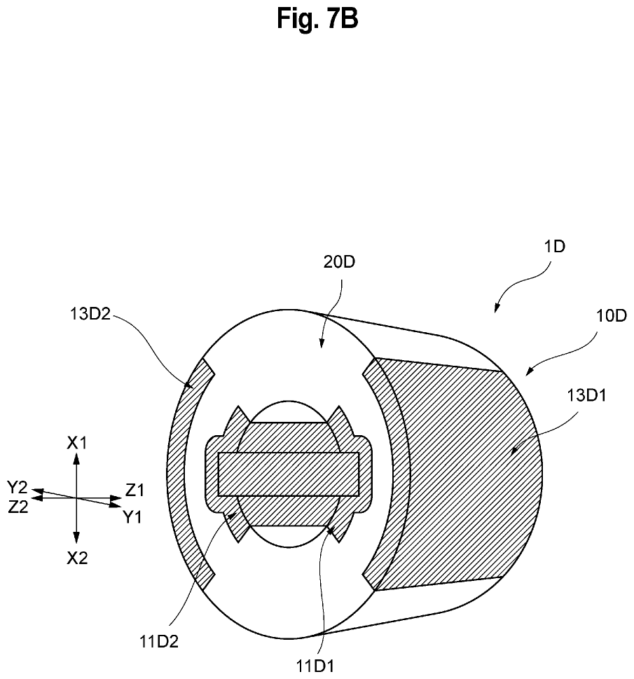

FIG. 7A is a diagram showing an example of the appearance of an ear tip 1D in the YZ plane according to the modification. In the example shown in FIG. 7A, contact portions 13D1 and 13D2 are provided on both ends in the Y direction with respect to a first member 10D of the ear tip 1D. The contact portions (e.g., extended portions) 13D1 and 13D2 are conductive elastic materials, but are insulated respectively, and a non-conductive second member 20D (e.g., silicone or urethane) is provided between both contact portions.

FIG. 7B is a perspective view of the ear tip 1D according to the modification. In the example shown in FIG. 7B, the contact portions 13D1 and 13D2 are located outside of the second member 20D. The first portion 13D1 out of the contact portions is conductively connected to a first portion 11D1 of the cylindrical portion, and the second portion 13D2 out of the contact portion is conductively connected to a second portion 11D2 of the cylindrical portion. The first portions (13D1 and 11D1) and the second portions (13D2 and 11D2) of the contact portions and cylindrical portion are electrically insulated, wherein one may be used as a bioelectrode for mainly detecting a biological signal and the other as a bioelectrode for detect a reference signal. In this case, the biosensor of the earphone body portion 2 is configured to output a differential signal, which is the biological signal minus the reference signal, to the outside. The first member 10D and the second member 20D may be detachable separate members or integrally molded.

### Second Embodiment

Next, an earphone 100 using the ear tips described in the first embodiment will be described. An ear tip according to a second embodiment may be any of the ear tips described in the first embodiment, and will be described in the second embodiment using reference sign 272.

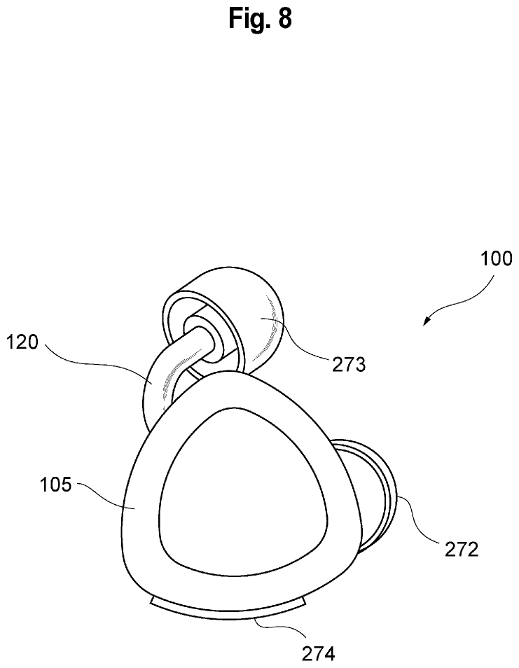

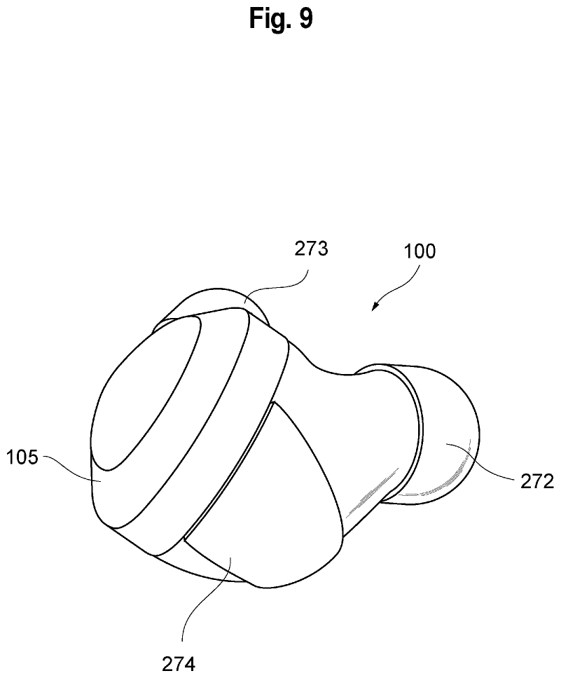

The components of the earphone 100 according to the second embodiment will be described with reference to FIGS. 8 and 9. FIGS. 8 and 9 are each a diagram showing an example of the earphone 100 according to the second embodiment. The earphone 100 shown in FIGS. 8 and 9 has three sensors. For example, the earphone 100 includes a main sensor 272 (first sensor), a reference sensor 273 (second sensor), and a ground sensor 274 (third sensor), which correspond to the ear tips according to the first embodiment.

The main sensor 272 is provided at a position where first biological information of the user can be acquired as an electrical signal. The location of the main sensor 272 is the tip portion that is inserted into the ear canal and adheres to the inner wall of the ear canal, as described in the first embodiment. The main sensor 272 outputs the sensed first biological information to an amplifier (amplifier) described below.

The reference sensor 273 is provided at a position where second biological information of the user can be acquired as an electrical signal. The location of the reference sensor 273 is, for example, at a tip of a wing 120. The reference sensor 273 outputs the sensed second biological information to the amplifier (amplifier) described below.

Here, the wing 120 is provided on a peripheral surface of a cover portion 105 in which a circuit board and the like are stored. The wing 120 protrudes from the peripheral surface of the cover portion 105 in an abbreviated U-shape toward the main sensor (ear tip) 272. The reference sensor 273 provided at the end of the wing 120 functions to hook onto the outer ear of the wearer when the earphone 100 is worn, helping to prevent the earphone 100 from falling out of the ear canal of the wearer. In addition, the reference sensor 273 comes into contact with the ear canal and is capable of measuring a second biological signal. The wing 120 can be made of an elastic and flexible material, as can a housing, which is an outer member of the earphone body portion 2. The housing may be formed of a non-conductive elastic material.

The ground sensor 274 is a sensor that acquires ground potential information as an electrical signal. The ground sensor 274 is positioned, for example, on the cover portion 105 side of the housing, in the opposite direction from the reference sensor 273. This is because the distance between the reference sensor 273 and the ground sensor 274 should be as far apart as possible. By separating the sensors, the accuracy of the biological signals acquired from the respective sensors can be improved. The ground sensor 274 outputs the sensed ground potential information to an A/D converter described below. The ground sensor 274 may have a convex shape protruding toward the outside, to facilitate adherence to the ear.

The material or the quality of the material of the main sensor 272 are as described in the first embodiment. The reference sensor 273 and the ground sensor 274 are made of, for example, conductive rubber, which contains silver or silver chloride. Preferably, to ensure proper conductivity, the silver or silver chloride contains a predetermined mass percentage or more of a conductive material in the conductive rubber.

The reference sensor 273 and the ground sensor 274 may be formed of a silicon material containing a metallic filler. For example, the reference sensor 273 and the ground sensor 274 can be made of a highly conductive material by appropriately blending silver, copper, gold, aluminum, zinc, nickel, or the like as a metallic filler into the silicone material. It is not necessary that all of the filler contained be silver or silver chloride, but only that a part of the filler be silver or silver chloride. This reduces the silver or silver chloride content, thus reducing the hardness of the rubber and creating conductive rubber having moderate hardness.

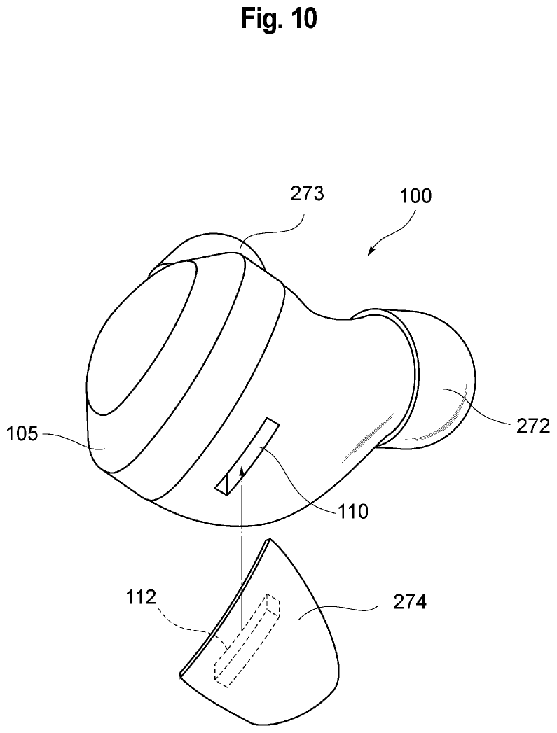

FIG. 10 is a diagram showing an example of the ground sensor 274 according to the second embodiment. In the example shown in FIG. 10, the ground sensor 274 is detachable from a predetermined area of the earphone body portion 2. For example, the ground sensor 274 has a convex-shaped second mounting structure 112, and the housing has a concave-shaped first mounting structure 110 that fits into the second mounting structure 112. When the second mounting structure 112 and the first mounting structure 110 are fitted together, their respective connection points make contact and are electrically connected, and the installation potential information from the ground sensor 274 is output to the A/D converter in the earphone body portion 2. The concavity-convexity relationship may be reversed.

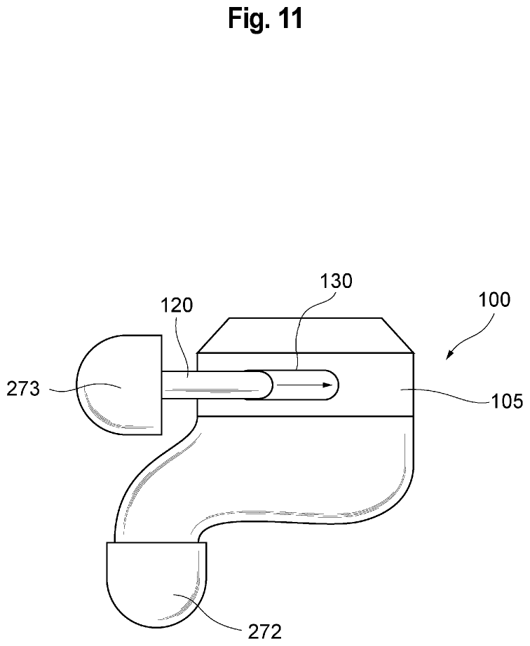

FIG. 11 is a diagram showing an example of the reference sensor 273 according to the second embodiment. In the example shown in FIG. 11, the wing 120 including the reference sensor 273 can slide along the circumferential direction of the cover portion 105. For example, a sliding mechanism 130 is provided in the circumferential direction of the cover portion 105, and the opposite end of the wing 120 from the reference sensor 273 configures a part of the sliding mechanism 130. This allows the position of the wing 120 to be adjustable with respect to the peripheral surface of the cover portion 105, so that the reference sensor 273 can be more in contact with the ear of the wearer and the second biological signal can be more appropriately acquired. The sliding mechanism 130 can move easily in the direction closer to the ear, but may apply more force than in the direction away from the ear.

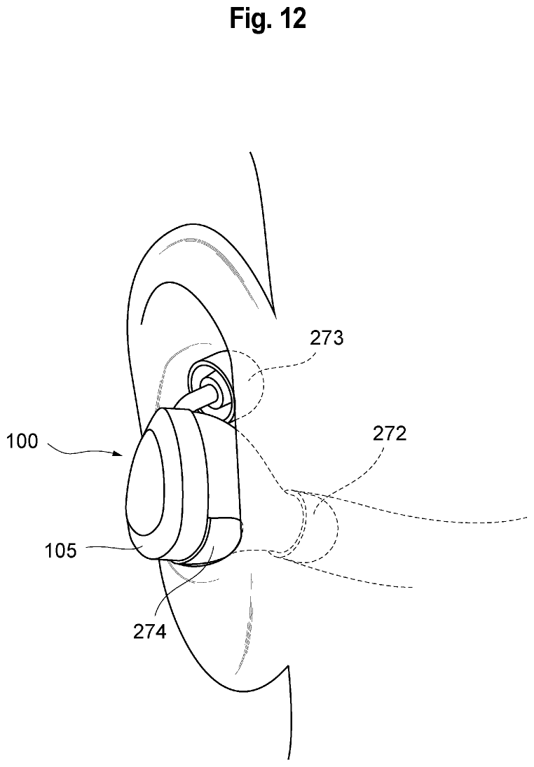

FIG. 12 is a diagram for explaining how three sensors of the earphone 100 according to the second embodiment come into contact with the wearer. As shown in FIG. 12, the main sensor 272 corresponding to the ear tip of the earphone 100 enters the ear canal, which causes the main sensor 272 to come into closer contact with the main sensor. Also, as the main sensor 272 enters the ear canal farther, the reference sensor 273 comes into further contact with the cavum conchae of the upper part of the concha auriculae of the wearer, and the ground sensor 274 comes into further contact with the cavum conchae of the lower part of the concha auriculae of the wearer.

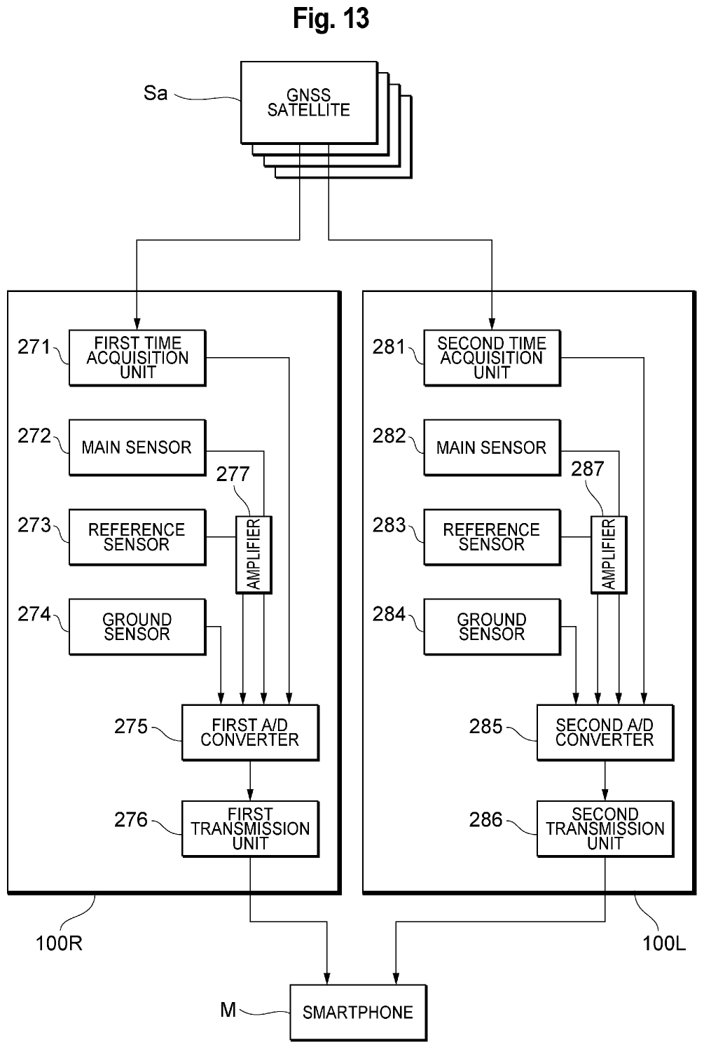

FIG. 13 is a diagram for explaining an overview of the earphone 100 according to the second embodiment. The earphone 100 according to the second embodiment shown in FIG. 13 includes a first earphone 100R and a second earphone 100L. The first earphone 100R is worn on the right ear of the user (wearer). The second earphone 100L is worn on the left ear of the user. The first earphone 100R and the second earphone 100L are configured to be able to communicate with a smartphone M. The smartphone M is an example of a communication terminal. The first earphone 100R and the second earphone 100L are configured to be able to receive GNSS signals transmitted from GNSS (Global Navigation Satellite System) satellites Sa, Sb.

The first earphone 100R has, as components, a first time acquisition unit 271, the main sensor 272 (first sensor), the reference sensor 273 (second sensor), the ground sensor 274 (third sensor), a first A/D converter 275, a first transmission unit 276, and an amplifier 277.

The first time acquisition unit 271 receives GNSS signals transmitted from the GNSS satellite Sa and acquires absolute time information contained in the GNSS signals. The first time acquisition unit 271 outputs the acquired absolute time information to the first A/D converter 275. The first time acquisition unit 271 includes, for example, a GPS (Global Positioning System) chip.

The main sensor 272 is provided at a position where first biological information of the user can be acquired as an electrical signal. The main sensor 272 outputs the sensed first biological information to the amplifier 277.

The reference sensor 273 is provided at a position where second biological information of the user can be acquired as an electrical signal. The reference sensor 273 outputs the sensed second biological information to the amplifier 277.

The ground sensor 274 is a sensor that acquires ground potential information as an electrical signal. The ground sensor 274 outputs the sensed ground potential information to the first A/D converter 275.

The amplifier 277 amplifies the first biological signal sensed by the main sensor 272 and the second biological signal sensed by the reference sensor 273, to expand the signal. The amplifier 277 outputs each signal obtained after expansion, to the first A/D converter 275.

The first A/D converter 275 samples each type of information in accordance with the timing of the absolute time information. For example, as a method of aligning sampling with the timing of the absolute time information, sampling may be performed in accordance with the absolute time information every time, or sampling may be performed in accordance with the absolute time information at regular intervals such as every second. The first A/D converter 275 outputs each type of sampled information to the first transmission unit 276.

The first transmission unit 276 transmits the first biological information, the second biological information, and the third biological information sampled by the first A/D converter 275 to the communication terminal M, in association with the absolute time information. The first transmission unit 276 may also generate first differential information, which is the difference between the first biological information output from the main sensor 272 and the ground potential information output from the ground sensor 274, and transmit the first differential information to the communication terminal M, in association with the absolute time information. Similarly, the first transmission unit 276 may generate second differential information, which is the difference between the second biological information output from the reference sensor 273 and the ground potential information output from the ground sensor 274, and transmit the second differential information to the communication terminal M, in association with the absolute time information. Associating the absolute time information with each type of information (e.g., adding a time stamp) may be performed by the first A/D converter 275.

In the example described above, the first transmission unit 276 associates the first biological information or the first differential information with the absolute time information and transmits the information to the communication terminal M on a first channel. The absolute time information associated with the first biological information or the first differential information is synchronized with the timing sensed by the main sensor 272. The first transmission unit 276 also associates the second biological information or the second differential information with the absolute time information and transmits it to the communication terminal M on a second channel. The absolute time information associated with the second biological information or the second differential information is synchronized with the timing sensed by the reference sensor 273. The second channel may be the same as or different from the first channel.

The second earphone 100L has the same configuration as the first earphone 100R. For the respective configurations in the second earphone 100L, those with the same names as the configurations in the first earphone 100R perform the same processing; thus the description thereof will be omitted accordingly.

The first transmission unit 276 transmits the first biological information of the right ear side wearing a first earpiece 2R to the communication terminal, with the absolute time information associated therewith, so that the communication terminal can accurately understand at which time the biological information is acquired.

Since the first transmission unit 276 transmits the first biological information of the right ear side wearing the first earphone 100R to the communication terminal, with the absolute time information associated therewith, the communication terminal can accurately understand at which time the biological information is acquired. Since earphones are worn on the ear, it is difficult to improve the accuracy of biological information even if a plurality of sensors for acquiring biological information are installed in the same earphones, because the potential difference of the acquired signals is small and the signals cancel each other out. On the other hand, by installing the sensors in respective different earphones and acquiring biological information by each sensor, the problem of not being able to ensure the potential difference can be solved. However, if the biological information acquired by different earphones are transmitted to the communication terminal for information processing, a new problem arises: errors may occur due to communication delays. In the second embodiment, therefore, the biological information acquired by each earphone is associated with the absolute time information, which is then transmitted to the communication terminal, thereby eliminating the errors caused by communication delays and making it possible to acquire accurate the biological information that ensures potential differences.

Although the present embodiment uses the absolute time information contained in a GNSS signal as an example of reference time information, other time information can be used as the reference time information as long as it has the accuracy necessary to identify the time of the biological information acquired by each earphone. For example, the first time acquisition unit 271 and the second time acquisition unit 281 may acquire signals such that the error of the reference time for each earphone is 1 ms or less. The reference time information can also be used as information that indicates the acquisition time of the biological information and is synchronized with the biological information acquired by the other earphones. The reference time information can be substituted for synchronization information to be synchronized with the biological information acquired by the other earphones. The positional relationship of each sensor may be exchanged or changed as appropriate.

The present embodiment has been described above with reference to specific examples. However, the present disclosure is not limited to these specific examples. Design changes made by those skilled in the art to these specific examples are also included in the scope of the present disclosure as long as they have the features of the present disclosure. The elements provided in each of the aforementioned specific examples, as well as their arrangement, conditions, shapes, and the like, are not limited to those shown in the examples, but can be changed as necessary. The elements provided in each of the aforementioned specific examples can be combined as needed as long as there is no technical inconsistency.

### REFERENCE SIGNS LIST

1 Ear tip
10 First member
11 Cylindrical portion
12 Tip
13 Extended portion
14 Mounting structure
15 Hollow portion
16 Slit
20 Second member
100 Earphone
105 Cover portion
110 First mounting structure
112 Second mounting structure
130 Sliding mechanism
272 Main sensor
273 Reference sensor
274 Ground sensor

1. An ear tip, comprising:
a conductive first member having a cylindrical portion having a first end and a second end, and a contact portion extending from the first end to the second end side; and
an elastic second member covering the outside of the cylindrical portion, wherein
the contact portion is located on the outside of the second member.

2. The ear tip according to claim 1, wherein the first member is formed using a silicon material containing a metallic filler.

3. The ear tip according to claim 1, wherein the second member is removably attached to the first member.

4. The ear tip according to claim 1, wherein the contact portion includes a first portion that is formed into a dome shape from the first end, and one or more second portions that extend from a predetermined position of the first portion to the second end side.

5. The ear tip according to claim 4, wherein a slit is provided between the plurality of second portions.

6. The ear tip according to claim 1, wherein the contact portion is provided in plurality, and at least two of the contact portions are electrically insulated.

7. An ear tip component, comprising:
a cylindrical portion having a first end and a second end; and
a contact portion extending from the first end to the second end side, wherein
the cylindrical portion and the contact portion are formed of a conductive material.

8. An earphone corresponding to the ear tip according to claim 1, and comprising:
a first sensor that acquires first biological information;
a second sensor that acquires second biological information at a position different from that of the first sensor; and
a third sensor that acquires third biological information at a position different from those of the first sensor and the second sensor.

9. The earphone according to claim 8, wherein
the second sensor comes into contact with a cavum conchae of an upper part of a concha auriculae of a wearer of the earphone, and
the third sensor comes into contact with a cavum conchae of a lower part of a concha auriculae of the wearer.

10. The earphone according to claim 8, wherein the second sensor is provided at a tip of a wing protruding from a body of the earphone, and a position of the wing is adjustable with respect to the body of the earphone.

11. The earphone according to claim 8, wherein the third sensor is detachable from a housing of the earphone.
